(19) United States
(12) Patent Application Publication
(10) Pub. No.: US 2022/0304777 A1
(43) Pub. Date: Sep. 29, 2022

(54) PATIENT-SPECIFIC RAPID MAXILLARY EXPANDER

(71) Applicants: INTER-MEDI CO., LTD., Jeollanam-go (KR); INDUSTRY FOUNDATION OF CHONNAM NATIONAL UNIVERSITY, Gwangju (KR)

(72) Inventors: Taek Rim YOON, Gwangju (KR); Ji Hye YOON, Gwangju (KR); Gyeong Min LEE, Gwangju (KR)

(21) Appl. No.: 17/056,122

(22) PCT Filed: Oct. 16, 2020
(86) PCT No.: PCT/KR2020/014200

§ 371 Date: Nov. 17, 2020

(30) Foreign Application Priority Data
Sep. 7, 2020 (KR) 10-2020-0114086

(51) Int. Cl. A61C 7/10; A61C 7/00

(52) U.S. Cl. CPC A61C 7/10; A61C 7/002

(57) ABSTRACT

A rapid maxillary expander according to an embodiment of the present invention has a structure in which teeth pressing portions are in surface contact with the inner surfaces of teeth and press the teeth outward, instead of a structure in which the teeth pressing portions surround the entire portions of the teen and the gums. Accordingly, a treatment operation may be facilitated, and the foreign body sensation of a patient may be reduced. In addition, since the teeth pressing portions are not exposed to the outside of the teeth, the aesthetic appearance of the patient is not impaired.

### TECHNICAL FIELD

The present disclosure relates to a rapid maxillary expander. More particularly, the present disclosure relates to a rapid maxillary expander having a structure in which teeth pressing portions are in surface contact with the inner surfaces of teeth and press the teeth outward, instead of a structure in which the teeth pressing portions surround the entire portions of the teen and the gums. Accordingly, a treatment operation may be facilitated, and the foreign body sensation of a patient may be reduced. In addition, since the teeth pressing portions are not exposed to the outside of the teeth, the aesthetic appearance of the patient is not impaired.

### BACKGROUND ART

The jawbones consist of the upper jawbone and the lower jawbone. According to the structure the jawbones, the upper teeth and the lower teeth are located on the upper and lower portions of the jawbones and are connected to the upper jawbone and the lower jawbone, respectively. In response to the movement of the jaw joint, the upper teeth and the lower teeth are aligned together to chew food.

In normal persons, the upper jawbone protrudes more forward than the lower jawbone. However, in some cases, the upper jawbone, which is smaller than the lower jawbone, may cause a malocclusion in which the upper jawbone and the lower jawbone are defectively aligned.

In the case of a malocclusion, an orthodontic treatment of moving the upper jawbone forward and expanding the upper jaw, in which the upper teeth are located, is conducted. A dental arch expander is a device used in an orthodontic treatment of a malocclusion patient having the upper jawbone less developed than the lower jawbone. The dental arch expander serves to expand the upper jawbone in order to facilitate the orthodontic treatment.

FIG. 1 is a view illustrating a dental arch expander 10 of the related art.

The dental arch expander 10 of the related art is a device fixed to the maxillary arch. The dental arch expander 10 includes a pair of bodies 11, arms fixed to the bodies 11, an expansion screw 13 adjusting the distance between the bodies 11, guide rails 14 guiding the movement of the bodies 11 when adjusting the distance between the bodies 11, a keyhole 15 formed in the expansion screw 13 and allowing a key to be inserted thereinto and rotated so as to rotate the expansion screw 13, and teeth pressing portions 16 coupled to the arms 12 and fixed to the teeth of the patient. The key is fitted into the keyhole 13 and is turned to rotate the expansion screw so that the bodies 11 are moved toward the teeth on both sides thereof 13, thereby increasing the distance between the bodies 11. In this manner, the dental arch expander expands the fixed upper jaw.

However, since the dental arch expander 10 of the related art is configured such that the teeth pressing portions 16 surround the entire portions of the teeth and the gums, a patient wearing the dental arch expander has a foreign body sensation, which is problematic. In addition, an aesthetic appearance may be impaired since the fixing portions of the dental arch expander are exposed to the outside of the teeth. Furthermore, the fabrication process of the dental arch expander is complicated, and the fabrication of the dental arch expander takes a long time.

### DISCLOSURE

### Technical Problem

Accordingly, the present disclosure has been made keeping in mind the above problems occurring in the prior art, and an objective of the present disclosure is to provide rapid maxillary expander having a structure in which teeth pressing portions are in surface contact with the inner surfaces of teeth and press the teeth outward, instead of a structure in which the teeth pressing portions surround the entire portions of the teeth and the gums. Accordingly, the foreign body sensation of a patient may be reduced and, since the teeth pressing portions are not exposed to the outside of the teeth, the aesthetic appearance of the patient is not impaired.

The objectives of the present disclosure are not limited to the aforementioned description, and other objectives not explicitly disclosed herein will be clearly understood by those skilled in the art from the description provided hereinafter.

### Technical Solution

In order to accomplish the above objective, provided is a rapid maxillary expander for expanding an upper jawbone. The rapid maxillary expander may include: a pair of expander bodies including a first expander body and a second expander body; an expansion screw, with one portion thereof being fastened to the first expander body, the other portion thereof being fastened to the second expander body, and a keyhole being provided in a central portion thereof, wherein the expansion screw adjusts a distance between the first expander body and the second expander body when rotated; a pair of guide rods including a first guide rod serving to guide a lateral movement of the second expander body, with one portion thereof being fitted into the first expander body, and the other portion thereof being exposed to outside of the second expander body, and a second guide rod serving to guide a lateral movement of the first expander body, with one portion thereof being fitted into the second expander body, and the other portion thereof being exposed to outside of the first expander body; a pair of connectors including a first connector, with one end thereof being connected to the first expander body and the other end thereof extending toward corresponding teeth, and a second connector, with one end thereof being connected to the second expander body and the other end thereof extending toward the teeth; and a pair of teeth pressing portions including a first teeth pressing portion connected to the other end of the first connector to press the corresponding teeth and a second teeth pressing portion connected to the other end of the second connector to press the corresponding teeth. One surface of the first teeth pressing portion and one surface of the second teeth pressing portion may have shapes corresponding to inner surfaces of the teeth in order to press the inner surfaces of the teeth outward.

The teeth pressing portions may be detachably attached to the connectors, respectively.

The connectors may be detachably attached to the expander bodies, respectively.

The teeth pressing portions may be fabricated by 3D printing or CAD or CAM processing, in accordance with 3D shape data of an internal portion of the dental arch.

The teeth pressing portions may be made of polymethyl methacrylate (PMMA), polyamide 12 (PA12), or a polymer, and are attached to the corresponding teeth via a resin.

### Advantageous Effects

The present disclosure has advantageous effects as follows.

In the rapid maxillary expander according to the present disclosure, the teeth pressing portions have a structure in surface contact with the inner surfaces of the teeth and pressing the teeth outward, instead of a structure surrounding the entire portions of the teeth and the gums. Thus, the foreign body sensation of the patient may be reduced. Since the teeth pressing portions are not exposed to the outside of the teeth, the aesthetic appearance of the patient is not impaired.

In addition, in the rapid maxillary expander according to the present disclosure, the teeth pressing portions are configured to be detachable from the connectors, or the connectors are configured to be detachable from the expander bodies. When a product is damaged, for example, when the teeth pressing portions are deformed, the expander may be used by selectively replacing the connector(s) or the teeth pressing portion(s) instead of replacing the entire product.

In addition, in the rapid maxillary expander according to the present disclosure, the teeth pressing portions are fabricated by 3D printing or CAD/CAM processing on the basis of 3D shape data of the interior of the dental arch. Thus, the teeth pressing portions may be easily fabricated into a structure customized to the shape of the teeth or the gums of the patient. In addition, since the teeth pressing portions are made of polymethyl methacrylate (PMMA), polyamide 12 (PA12), or any other polymer, there is an advantage in that the teeth pressing portions may be easily bonded to teeth.

### DESCRIPTION OF DRAWINGS

FIG. 1 is a view illustrating a dental arch expander of the related art;

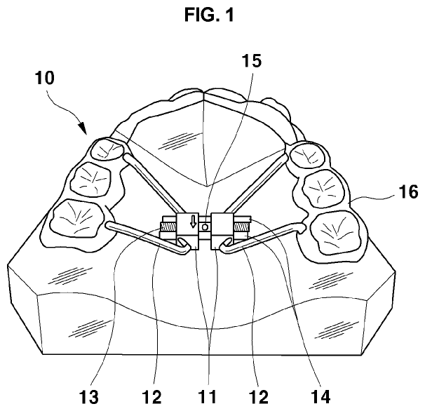

FIG. 2 is a view illustrating an application of a rapid maxillary expander according to an embodiment of the present disclosure;

FIG. 3 is an exploded perspective view illustrating the rapid maxillary expander according to the embodiment of the present disclosure; and

FIG. 4 is a view illustrating connectors of a rapid maxillary expander according to another embodiment of the present disclosure.

110: expander body
111: first expander body
112: second expander body
120: expansion screw
130: guide rod
131: first guide rod
132: second guide rod
140: connector
141: first connector
142: second connector
150: teeth pressing portion
151: first teeth pressing portion
152: second teeth pressing portion

### BEST MODE

The terms used in the present disclosure are selected to be general terms that are widely used at present. In particular cases, some terms are arbitrarily selected by the applicant, and such terms should be interpreted in consideration of the meaning described or used in the detailed description of the present disclosure rather than as common definitions of the terms.

Hereinafter, technical configurations of the present disclosure will be described in detail with reference to exemplary embodiments illustrated in the accompanying drawings.

However, the present disclosure is not limited to the embodiments to be described hereinafter but may be embodied in a variety of different forms. Throughout the specification, the same elements are designated by the same reference numerals.

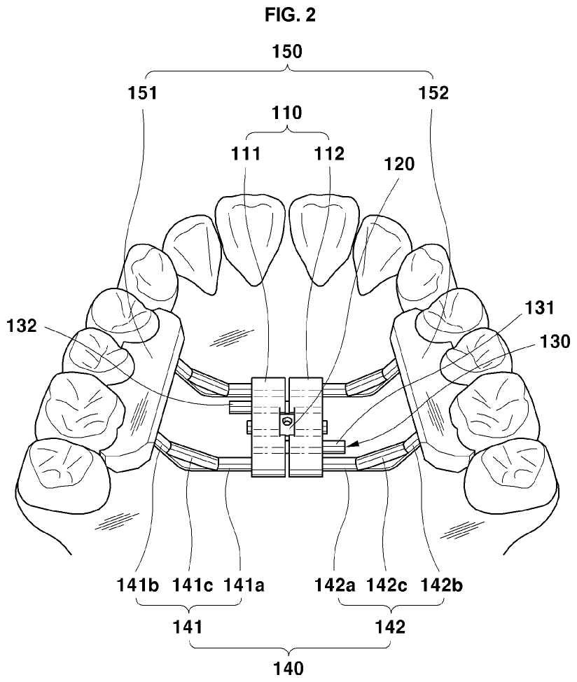

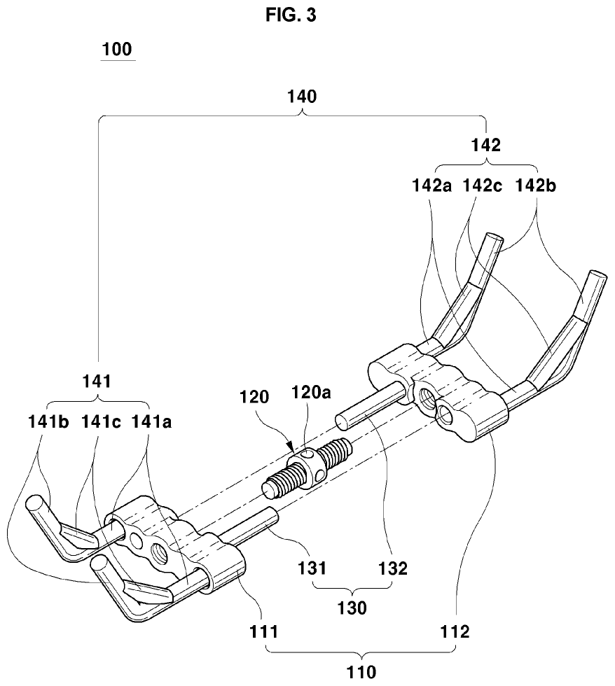

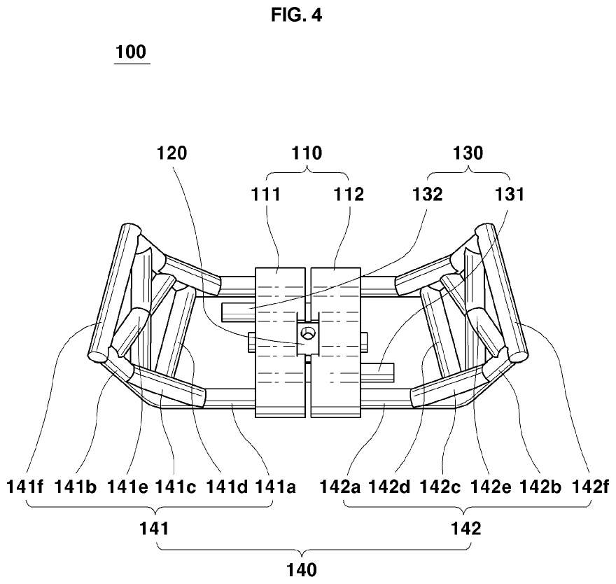

FIG. 2 is a view illustrating an application of a rapid maxillary expander according to an embodiment of the present disclosure, FIG. 3 is an exploded perspective view illustrating the rapid maxillary expander according to the embodiment of the present disclosure, and FIG. 4 is a view illustrating connectors of a rapid maxillary expander according to another embodiment of the present disclosure.

Referring to FIGS. 2 and 3, a rapid maxillary expander 100 is used in an orthodontic treatment of a malocclusion patient having the upper jawbone less developed than the lower jawbone, and serves to expand the upper jawbone to facilitate the orthodontic treatment. The rapid maxillary expander 100 includes expander bodies 110, an expansion screw 120, guide rods 130, connectors 140, and teeth pressing portions 150.

The expander bodies 110 are a pair of expander bodies including a first expander body 111 and a second expander body 112, each of which has an expansion screw hole extending through the central portion in the axial direction.

In addition, each of the expander bodies 110 may have first and second guide rod holes on both sides of the expansion screw hole and first and second connector holes in outermost portions thereof on both sides of the expansion screw hole.

The expansion screw 120 has a keyhole 120a extending in a top-bottom direction through the central portion thereof. One portion of the expansion screw 120 is fastened to the first expander body 111, while the other portion of the expansion screw 120 is fastened to the second expander body 112.

Thus, when the expansion screw 120 rotates, the first expander body 111 and the second expander body 112 are driven so that the distance therebetween gradually increases.

The guide rods 130 are a pair of guide rods 130 including a first guide rod 131 and a second guide rod 132.

Here, the first guide rod 131 serves to guide the lateral movement of the second expander body 112, with one portion thereof being fitted into the first expander body 111, and the other portion thereof being exposed to the outside of the second expander body 112.

In addition, the second guide rod 132 serves to guide the lateral movement of the first expander body 111, with one portion thereof being fitted into the second expander body 112, and the other portion thereof being exposed to the outside of the first expander body 111.

The connectors 140 are a pair of connectors including a first connector 141 and a second connector 142.

Here, the first connector 141 is configured such that one end thereof is connected to the first expander body 111 and the other end thereof extends toward the teeth.

For example, the first connector 141 may include a pair of first bar portions 141a, a pair of second bar portions 141b, and a pair of third bar portions 141c.

Here, each of the first bar portions 141a is configured such that one end thereof is fitted into the first expander body 111 so as to be parallel to the first expander body 111.

In addition, each of the second bar portions 141b is provided at a predetermined angle with respect to the corresponding first bar portion 141a such that one end thereof is integrally connected to the other end of the first bar portion 141a and the other end thereof extends toward the teeth.

In addition, each of the third bar portions 141c is configured such that one end thereof is integrally connected to the central portion of the corresponding first bar portion 141a and the other end thereof is integrally connected to the central portion of the corresponding second bar portion 141b. With this configuration, the third bar portions 141c serve to increase the strength of the connectors.

In addition, the second connector 142 is configured such that one end thereof is connected to the second expander body 112 and the other end thereof extends toward the teeth.

In addition, like the first connector 141, the second connector 142 includes a pair of first bar portions 142a, a pair of second bar portions 142b, and a pair of third bar portions 142c. Since the bar portions 142a, 142b, and 142c correspond to the bar portions 141a, 141b, and 141c of the first connector 141, repetitive descriptions thereof will be omitted.

FIG. 4 is a view illustrating connectors of a rapid maxillary expander according to an embodiment of the present disclosure.

Referring to FIG. 4, the connectors 140 are a pair of connectors including a first connector 141 and a second connector 142. The first connector 141 may include a pair of first bar portions 141a, a pair of second bar portions 141b, a pair of third bar portions 141c, a fourth bar portion 141d, fifth bar portions 141e, and a sixth bar portion 141f.

Since the first bar portions 141a, the pair of second bar portions 141b, and the pair of third bar portions 141c are the same as those described above, repetitive descriptions thereof will be omitted.

The fourth bar portion 141d connects the pair of first bar portions 141a. The fifth bar portions 141e are a pair of bar portions arranged in an X-shape and connecting the first bar portions 141a and the second bar portions 141b disposed in symmetric positions. The sixth bar portion 161f connects the pair of second bar portions 142b in order to further increase the strength of the connector.

In addition, the second connector 142 includes a pair of first bar portions 142a, a pair of second bar portions 142b, a pair of third bar portions 142c, a fourth bar portion 142d, fifth bar portions 142e, and a sixth bar portion 142f. Since these bar portions correspond to those of the first connector 141, repetitive descriptions thereof will be omitted.

In addition, the connectors 141 and 142 may be configured to be detachably attached to the expander bodies 111 and 112, respectively.

In other words, the first connector 141 is provided to be detachable from the first expander body 111, while the second connector 132 is provided to be detachable from the second expander body 112.

According to this configuration, when the teeth pressing portions are deformed or a portion or the entirety of the connectors is damaged, the expander may be used by selectively replacing the connector or connectors instead of replacing the entire product.

The teeth pressing portions 150 are a pair of teeth pressing portions including a first teeth pressing portion 151 and a second teeth pressing portion 152.

Here, the first teeth pressing portion 151 is connected to the other end of the first connector 141 to press the teeth on one side, while the second teeth pressing portion 152 is connected to the other end of the second connector 142 to press the teeth on the other side.

In addition, one surface of the first teeth pressing portion 151 and one surface of the second teeth pressing portion 152 have shapes corresponding to inner surfaces of the teeth in corresponding positions in order to press the inner surfaces of the teeth in directions from inside to outside.

That is, the teeth pressing portions 150 have a structure in surface contact with the inner surfaces of the teeth and pressing the teeth outward, instead of a structure surrounding the entire portions of the teeth and the gums. Thus, the foreign body sensation of the patient may be reduced. Since the teeth pressing portions are not exposed to the outside of the teeth, the aesthetic appearance of the patient is not impaired.

In addition, the teeth pressing portions 150 may be provided to be detachably attached to the connectors 141 and 142, respectively.

According to this configuration, when any of the teeth pressing portions 150 is deformed or damaged, the expander may be used by selectively replacing the teeth pressing portion(s) 150 instead of replacing the entire product.

In addition, the teeth pressing portions 150 may be fabricated by three-dimensional (3D) printing or computer aided design (CAD) or computer aided manufacturing (CAM) processing, on the basis of 3D shape data of the interior of the dental arch. The teeth pressing portions 150 may made of polymethyl methacrylate (PMMA), polyamide 12 (PA12), or any other polymer, and may be attached to teeth via a resin.

That is, since the teeth pressing portions 150 are fabricated by 3D printing or CAD/CAM processing on the basis of 3D shape data of the interior of the dental arch, the teeth pressing portions 150 may be easily fabricated into a structure customized to the shape of the teeth or the gums of the patient. In addition, since the teeth pressing portions 150 are made of polymethyl methacrylate (PMMA), polyamide 12 (PA12), or any other polymer, there is an advantage in that the teeth pressing portions 150 may be easily bonded to teeth.

Although the exemplary embodiments of the present disclosure have been described for illustrative purposes, those skilled in the art will appreciate that various modifications, additions, and substitutions are possible, without departing from the scope and spirit of the present disclosure as disclosed in the accompanying claims.

### INDUSTRIAL APPLICABILITY

The present disclosure may be used in an orthodontic treatment of a malocclusion patient having the upper jawbone less developed than the lower jawbone, in order to expand the upper jawbone so that the orthodontic treatment may be easily conducted.

1. A rapid maxillary expander for expanding an upper jawbone, comprising:
a pair of expander bodies including a first expander body and a second expander body;
an expansion screw, with one portion thereof being fastened to the first expander body, the other portion thereof being fastened to the second expander body, and a keyhole being provided in a central portion thereof, wherein the expansion screw adjusts a distance between the first expander body and the second expander body when rotated;
a pair of guide rods including a first guide rod serving to guide a lateral movement of the second expander body, with one portion thereof being fitted into the first expander body, and the other portion thereof being exposed to outside of the second expander body, and a second guide rod serving to guide a lateral movement of the first expander body, with one portion thereof being fitted into the second expander body, and the other portion thereof being exposed to outside of the first expander body;
a pair of connectors including a first connector, with one end thereof being connected to the first expander body and the other end thereof extending toward corresponding teeth, and a second connector, with one end thereof being connected to the second expander body and the other end thereof extending toward the teeth; and
a pair of teeth pressing portions including a first teeth pressing portion connected to the other end of the first connector to press the corresponding teeth and a second teeth pressing portion connected to the other end of the second connector to press the corresponding teeth,
wherein one surface of the first teeth pressing portion and one surface of the second teeth pressing portion have shapes corresponding to inner surfaces of the teeth in order to press the inner surfaces of the teeth outward.

2. The rapid maxillary expander of claim 1, wherein the teeth pressing portions are detachably attached to the connectors, respectively.

3. The rapid maxillary expander of claim 1, wherein the connectors are detachably attached to the expander bodies, respectively.

4. The rapid maxillary expander of claim 1, wherein the teeth pressing portions are fabricated by 3D printing, computer-aided design (CAD) processing or computer-aided manufacturing (CAM) processing, in accordance with 3D shape data of an internal portion of the dental arch.

5. The rapid maxillary expander of claim 1, wherein the teeth pressing portions are made of polymethyl methacrylate (PMMA), polyamide 12 (PA12), or a polymer, and are attached to the corresponding teeth via a resin.
